(19) United States
(12) Patent Application Publication
(10) Pub. No.: US 2025/0195206 A1
(43) Pub. Date: Jun. 19, 2025

(54) SYSTEMS AND METHODS FOR TREATING SOFT TISSUE

(71) Applicants: Smith & Nephew, Inc., Memphis, TN (US); Smith & Nephew Orthopaedics AG, Zug (CH); Smith & Nephew Asia Pacific Pte. Limited

(72) Inventors: Carolyn M. Krasniak, Melrose, MA (US); David A. Yeung, Andover, MA (US); Brendan Jablonski, Boston, MA (US); Rachel S. Keen, Medford, MA (US); Kevin M. Falco, Chelmsford, MA (US)

(73) Assignees: Smith & Nephew, Inc., Memphis, TN (US); Smith & Nephew Orthopaedics AG, Zug (CH); Smith & Nephew Asia Pacific Pte. Limited, (omitted)

(21) Appl. No.: 19/073,436

(22) Filed: Mar. 7, 2025

Related U.S. Application Data
(63) Continuation of application No. PCT/US2023/032161, filed on Sep. 7, 2023.
(60) Provisional application No. 63/405,252, filed on Sep. 9, 2022.

(51) Int. Cl. A61F 2/08

(52) U.S. Cl. CPC A61F 2/0811; A61F 2002/0817; A61F 2002/0858; A61F 2002/0876; A61F 2210/0004

(57) ABSTRACT

Systems and methods for securing a sheet-like implant to soft tissue and/or bone at a treatment site may include a tissue anchor delivery device configured to deliver at least one tissue anchor to the treatment site. Some systems may include a tissue anchor deployment device separate from the tissue anchor delivery device and configured to be oriented non-parallel to the tissue anchor delivery device. In some systems, the tissue anchor delivery device may include a tubular deployment member having at least one flange extending radially inward to define a distal aperture having a minimum lateral extent less than a maximum lateral extend of an enlarged head of a tissue anchor. Some systems and methods may be configured to deploy at least one tissue anchor into the sheet-like implant at an oblique angle to an upper surface of the sheet-like implant.

### CROSS REFERENCE TO RELATED APPLICATIONS

This application is a continuation of International Application No. PCT/US2023/032161, filed on Sep. 7, 2023, which claims the benefit of and priority to U.S. Provisional Patent Application Ser. No. 63/405,252, filed on Sep. 9, 2022, the disclosures of which are incorporated herein by reference.

### TECHNICAL FIELD

The present disclosure pertains generally to medical devices and methods of using medical devices. More particularly, the present disclosure relates to medical devices and/or systems, and methods of using the same, for arthroscopic placement of a sheet-like implant over or in the area of a full or partial thickness tear of a tendon, such as the supraspinatus tendon of the shoulder.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. There is an ongoing need to deliver, position, and secure medical implants to soft tissue during an arthroscopic procedure in order to treat injuries to the rotator cuff, rotator cuff tendons, or other soft tissue or tendon injuries throughout a body.

### SUMMARY

In one example, a system for securing a sheet-like implant to soft tissue and/or bone at a treatment site may comprise a tissue anchor delivery device including an elongate shaft having a longitudinal axis and a plurality of tissue anchors movably disposed within the elongate shaft, and a tissue anchor deployment device separate from the tissue anchor delivery device, the tissue anchor deployment device oriented non-parallel to the elongate shaft of the tissue anchor delivery device and configured to engage a first tissue anchor of the plurality of tissue anchors proximate a distal end of the elongate shaft to insert the first tissue anchor into the sheet-like implant and the soft tissue and/or bone at the treatment site to secure the sheet-like implant thereto.

In addition or alternatively to any example described herein, the first tissue anchor is exposed to an exterior of the elongate shaft proximate the distal end of the elongate shaft.

In addition or alternatively to any example described herein, the first tissue anchor is disposed distal of the distal end of the elongate shaft.

In addition or alternatively to any example described herein, the tissue anchor deployment device includes an outer tube configured to engage the elongate shaft and an inner member configured to engage the first tissue anchor distal of the distal end of the elongate shaft.

In addition or alternatively to any example described herein, the elongate shaft includes an engagement feature configured to engage a mating engagement feature of the outer tube.

In addition or alternatively to any example described herein, the elongate shaft includes a side facing window through a wall of the elongate shaft.

In addition or alternatively to any example described herein, the tissue anchor deployment device includes an outer tube configured to engage the elongate shaft and an inner member configured to engage the first tissue anchor through the side facing window.

In addition or alternatively to any example described herein, the elongate shaft includes an engagement feature configured to engage a mating engagement feature of the outer tube.

In addition or alternatively to any example described herein, the plurality of tissue anchors each include a first prong, a second prong, and a bridge extending from the first prong to the second prong.

In addition or alternatively to any example described herein, the first prong and the second prong extend transverse to the longitudinal axis of the elongate shaft.

In addition or alternatively to any example described herein, the tissue anchor deployment device is configured to engage the bridge to insert the first prong and the second prong into the sheet-like implant and the soft tissue and/or bone.

In addition or alternatively to any example described herein, the elongate shaft includes a stop feature at the distal end of the elongate shaft, the stop feature being configured to prevent distal advancement of the distalmost one of the plurality of tissue anchors past a predetermined point.

In addition or alternatively to any example described herein, each of the plurality of tissue anchors is releasably attached to an adjacent one of the plurality of tissue anchors.

In addition or alternatively to any example described herein, a method of treating soft tissue at a joint region of a patient may comprise: creating a lateral portal and a superior portal through a patient's skin to access a treatment site at the joint region; deploying a sheet-like implant from an implant delivery device onto the soft tissue at the treatment site; inserting a tissue anchor delivery device through the lateral portal to the treatment site, the tissue anchor delivery device including an elongate shaft having a longitudinal axis and a plurality of tissue anchors movably disposed within the elongate shaft; inserting a tissue anchor deployment device non-parallel to the longitudinal axis of the elongate shaft through the superior portal to the treatment site; engaging the tissue anchor deployment device with a first tissue anchor of the plurality of tissue anchors proximate a distal end of the elongate shaft; and deploying the first tissue anchor into the sheet-like implant to secure the sheet-like implant to the soft tissue and/or bone at the treatment site using the tissue anchor deployment device.

In addition or alternatively to any example described herein, the plurality of tissue anchors each include a first prong, a second prong, and a bridge extending from the first prong to the second prong.

In addition or alternatively to any example described herein, the first prong and the second prong extend laterally relative to the longitudinal axis of the elongate shaft.

In addition or alternatively to any example described herein, the tissue anchor deployment device is configured to engage the bridge to exert a force on the tissue anchor to push the tissue anchor into the sheet-like implant and the soft tissue and/or bone.

In addition or alternatively to any example described herein, the method may further comprise after deploying the first tissue anchor, advancing the remaining plurality of tissue anchors distally within the elongate shaft to position a second tissue anchor proximate the distal end of the elongate shaft.

In addition or alternatively to any example described herein, the method may further comprise engaging the tissue anchor deployment device with the second tissue anchor of the plurality of tissue anchors proximate the distal end of the elongate shaft; and deploying the second tissue anchor into the sheet-like implant to secure the sheet-like implant to the soft tissue and/or bone at the treatment site using the tissue anchor deployment device.

In addition or alternatively to any example described herein, the tissue anchor deployment device is oriented generally perpendicular to the longitudinal axis of the elongate shaft during deployment of the first tissue anchor.

In addition or alternatively to any example described herein, a system for securing a sheet-like implant to tissue at a treatment site may comprise a tissue anchor delivery device including: a handle, and an elongate shaft extending distally from the handle along a longitudinal axis, the elongate shaft including an outer sleeve and a tubular deployment member slidably disposed within the outer sleeve; and a plurality of tissue anchors movably disposed within a lumen of the tubular deployment member. Each tissue anchor may include a stem extending longitudinally along the longitudinal axis, an enlarged head disposed at a proximal end of the stem, and one or more anchor barbs extending laterally from a distal portion of the stem. A distal portion of the tubular deployment member may include at least one flange extending radially inward from a wall of the tubular deployment member to define a distal aperture of the tubular deployment member. When the distal portion of the tubular deployment member is disposed within the outer sleeve, the distal aperture may have a minimum lateral extent measured transverse to the longitudinal axis that is less than a maximum lateral extent of the enlarged head measured transverse to the longitudinal axis.

In addition or alternatively to any example described herein, the distal portion of the tubular deployment member includes a plurality of deflectable strips extending proximally from a distal end of the tubular deployment member, the plurality of deflectable strips being movable radially between a first position and a second position, the first position being closer to the longitudinal axis than the second position.

In addition or alternatively to any example described herein, when the distal portion of the tubular deployment member is disposed within the outer sleeve, the plurality of deflectable strips are disposed in the first position.

In addition or alternatively to any example described herein, the plurality of deflectable strips are biased radially outwardly toward the second position when the distal portion of the tubular deployment member is unconstrained by the outer sleeve.

In addition or alternatively to any example described herein, actuation of a trigger member on the handle translates the outer sleeve proximally relative to the tubular deployment member to expose the plurality of deflectable strips.

In addition or alternatively to any example described herein, the plurality of tissue anchors are configured to advance distally relative to the tubular deployment member subsequent to exposing the plurality of deflectable strips.

In addition or alternatively to any example described herein, the tissue anchor delivery device includes a biasing element configured to advance the plurality of tissue anchors distally within the tubular deployment member when the distal portion of the tubular deployment member is unconstrained by the outer sleeve.

In addition or alternatively to any example described herein, when the distal portion of the tubular deployment member is disposed within the outer sleeve and the plurality of deflectable strips are disposed in the first position, and a leading one of the plurality of tissue anchors is disposed distal of the at least one flange, a distal force applied to the tubular deployment member is configured to insert the leading one of the plurality of tissue anchors into the sheet-like implant and soft tissue and/or bone.

In addition or alternatively to any example described herein, a distal surface of the distal end of the tubular deployment member is configured to engage the enlarged head of the leading one of the plurality of tissue anchors to insert the leading one of the tissue anchors into the sheet-like implant and soft tissue and/or bone.

In addition or alternatively to any example described herein, the outer sleeve extends distal of the distal end of the tubular deployment member when the distal force is initially applied to the tubular deployment member.

In addition or alternatively to any example described herein, application of the distal force to the tubular deployment member translates the tubular deployment member distally relative to the outer sleeve.

In addition or alternatively to any example described herein, the tissue anchor delivery device includes a shaft extending longitudinally within the lumen of the tubular deployment member. Each of the plurality of tissue anchors includes a central bore extending longitudinally within the stem. The shaft extends through the central bore of each of the plurality of tissue anchors.

In addition or alternatively to any example described herein, the at least one flange includes a tapered surface extending distally and radially inwardly toward the distal aperture.

In addition or alternatively to any example described herein, a method of treating soft tissue at a joint region of a patient may comprise: deploying a sheet-like implant from an implant delivery device onto the soft tissue at the treatment site; advancing a tissue anchor delivery device to the treatment site, the tissue anchor delivery device including a handle, and an elongate shaft extending distally from the handle, the elongate shaft including an outer sleeve and a tubular deployment member slidably disposed within the outer sleeve; wherein a plurality of tissue anchors are movably disposed within the lumen of the tubular deployment member, each tissue anchor including a stem extending longitudinally along a longitudinal axis of the elongate shaft, an enlarged head disposed at a proximal end of the stem, and one or more anchor barbs extending laterally from a distal portion of the stem; and applying a distal force to the tubular deployment member to deploy a leading one of the plurality of tissue anchors into the sheet-like implant and soft tissue and/or bone at the treatment site to secure the sheet-like implant thereto.

In addition or alternatively to any example described herein, a distal portion of the tubular deployment member includes at least one flange extending radially inward from a wall of the tubular deployment member to define a distal aperture of the tubular deployment member. When the distal portion of the tubular deployment member is disposed within the outer sleeve, the distal aperture has a minimum lateral extent measured transverse to the longitudinal axis that is less than a maximum lateral extent of the enlarged head measured transverse to the longitudinal axis.

In addition or alternatively to any example described herein, the distal portion of the tubular deployment member includes a plurality of deflectable strips extending proximally from a distal end of the tubular deployment member, the plurality of deflectable strips being movable radially between a first position and a second position, the first position being closer to the longitudinal axis than the second position.

In addition or alternatively to any example described herein, when the distal portion of the tubular deployment member is disposed within the outer sleeve, the plurality of deflectable strips are disposed in the first position.

In addition or alternatively to any example described herein, the plurality of deflectable strips are biased radially outwardly toward the second position when the distal portion of the tubular deployment member is unconstrained by the outer sleeve.

In addition or alternatively to any example described herein, the method may further comprise actuating a trigger member on the handle to translate the outer sleeve proximally relative to the tubular deployment member to expose the plurality of deflectable strips.

In addition or alternatively to any example described herein, the method may further comprise advancing the plurality of tissue anchors distally relative to the tubular deployment member after exposing the plurality of deflectable strips.

In addition or alternatively to any example described herein, the tissue anchor delivery device includes a biasing element configured to advance the plurality of tissue anchors distally within the tubular deployment member when the distal portion of the tubular deployment member is unconstrained by the outer sleeve.

In addition or alternatively to any example described herein, a distal surface of the distal end of the tubular deployment member is configured to engage the enlarged head of the leading one of the plurality of tissue anchors to insert the leading one of the plurality of tissue anchors into the sheet-like implant and soft tissue and/or bone.

In addition or alternatively to any example described herein, the outer sleeve extends distal of the distal end of the tubular deployment member when the distal force is initially applied to the tubular deployment member.

In addition or alternatively to any example described herein, application of the distal force to the tubular deployment member translates the tubular deployment member distally relative to the outer sleeve.

In addition or alternatively to any example described herein, a method of treating soft tissue may comprise: deploying a sheet-like implant from an implant delivery device onto the soft tissue at a treatment site in a shoulder of a patient; inserting a tissue anchor delivery device through a lateral portal in the shoulder to the treatment site; and deploying at least one tissue anchor from the tissue anchor delivery device into the sheet-like implant and the soft tissue at the treatment site at an oblique angle to an upper surface of the sheet-like implant in order to secure the sheet-like implant to the soft tissue.

In addition or alternatively to any example described herein, each tissue anchor includes a single axial prong, at least one anchoring barb extending laterally from a distal portion of the single axial prong, and a tissue securement arm extending laterally from a proximal portion of the single axial prong.

In addition or alternatively to any example described herein, the tissue securement arm includes a curved portion extending away from the single axial prong to a free end, wherein the free end extends distal of a proximalmost extent of the curved portion.

In addition or alternatively to any example described herein, after deploying the at least one tissue anchor from the tissue anchor delivery device into the sheet-like implant and the soft tissue, the free end of the tissue securement arm extends into the sheet-like implant.

In addition or alternatively to any example described herein, the tissue anchor delivery device includes a distal tine configured to pierce the soft tissue, and the at least one tissue anchor is disposable on the distal tine for deployment into the sheet-like implant and the soft tissue at the treatment site.

In addition or alternatively to any example described herein, the oblique angle is 60 degrees or less relative to the upper surface of the sheet-like implant.

In addition or alternatively to any example described herein, the method may further comprise inserting a bone anchor delivery device through the lateral portal to the treatment site; and deploying at least one bone anchor from the bone anchor delivery device to secure the sheet-like implant to bone adjacent the treatment site.

In addition or alternatively to any example described herein, a system for securing a sheet-like implant to soft tissue at a treatment site may comprise a tissue anchor delivery device including an elongate shaft and a distal tine extending distally from the elongate shaft, and a tissue anchor releasably disposable on the distal tine. The tissue anchor may be configured for insertion into the sheet-like implant and the soft tissue at the treatment site to secure the sheet-like implant to the soft tissue. The tissue anchor may include a single axial prong, at least one anchoring barb extending laterally from a distal portion of the single axial prong, and a tissue securement arm extending laterally from a proximal portion of the single axial prong.

In addition or alternatively to any example described herein, the tissue securement arm includes a curved portion extending away from the single axial prong to a free end, wherein the free end extends distal of a proximalmost extent of the curved portion.

In addition or alternatively to any example described herein, the free end of the tissue securement arm is configured to penetrate into the sheet-like implant.

In addition or alternatively to any example described herein, the tissue anchor is formed from a resorbable material. The above summary of some embodiments, aspects, and/or examples is not intended to describe each disclosed embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a stylized view of a shoulder of a patient having one or more portals or incisions formed therein;

FIG. 2 illustrates a shoulder including a head of the humerus mating with the glenoid fossa of the scapula at a glenohumeral joint and a sheet-like implant being affixed to a tendon using an implant delivery device;

FIG. 3 illustrates an example implant delivery device attached to the sheet-like implant;

FIG. 4 is a perspective view illustrating a bone anchor delivery device being used to deploy one or more bone anchors securing the sheet-like implant to the head of the humerus;

FIG. 5 illustrates selected aspects of a system for securing the sheet-like implant to soft tissue and/or bone at a treatment site;

FIGS. 6-7 illustrate selected aspects of the system of FIG. 5;

FIGS. 8-9 illustrates selected aspects of an alternative configuration of the system of FIG. 5;

FIG. 10 is a perspective view illustrating the sheet-like implant secured to soft tissue and/or bone at the treatment site;

FIG. 10A is a cross-sectional view of FIG. 10 taken along the line 10A-10A;

FIG. 11-14 illustrate selected aspects of a system and method for securing the sheet-like implant to tissue at the treatment site;

FIG. 15 illustrates selected aspects of an alternative configuration of the system of FIGS. 11-14;

FIG. 16 illustrates selected aspects of an alternative configuration of the system of FIGS. 11-14;

FIGS. 17-18 illustrate selected aspects of the system of FIGS. 11-14;

FIG. 19 illustrates selected aspects of a system for securing the sheet-like implant to tissue at the treatment site; and

FIGS. 20-22 illustrate selected aspects of a method for securing the sheet-like implant to tissue at the treatment site using the system of FIG. 19.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. Like reference numerals indicate like elements throughout the views. The detailed description and drawings are intended to illustrate but not limit the disclosure. Those skilled in the art will recognize that the various elements described and/or shown may be arranged in various combinations and configurations without departing from the scope of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include one or more particular features, structures, and/or characteristics. However, such recitations do not necessarily mean that all embodiments include the particular features, structures, and/or characteristics. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, individual elements described herein, even if not explicitly shown in a particular combination, are contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures generally illustrate selected components and/or arrangements of medical devices, systems, and/or methods. It should be noted that in any given figure, some features may not be shown, or may be shown schematically, for simplicity. Additional details regarding some elements may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to features or elements may be equally referred to all instances and quantities beyond one of said feature or element. As such, it will be understood that the following discussion may apply equally to any and/or all of the elements for which there are more than one within the medical devices, systems, and/or methods, unless explicitly stated to the contrary.

With its complexity, range of motion, and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon and/or promote tissue reformation. Therefore, there is an ongoing need to deliver, position, and secure medical implants to soft tissue during an arthroscopic procedure in order to treat injuries to the rotator cuff, rotator cuff tendons, or other soft tissue or tendon injuries throughout a body.

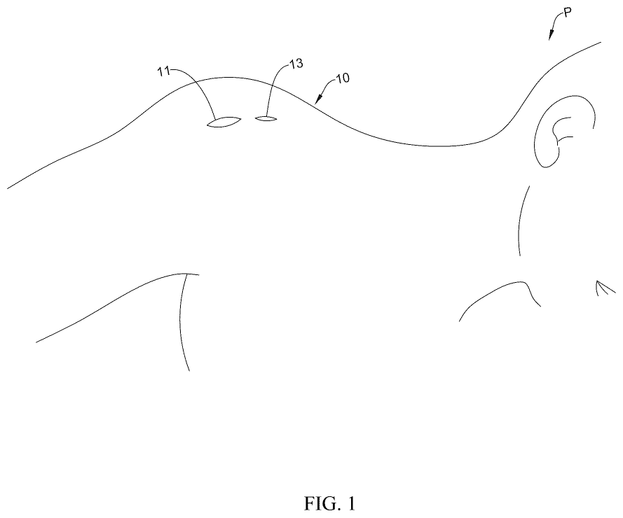

FIG. 1 is a stylized view of a shoulder 10 of a patient P. In some instances, delivery of a medical implant (e.g., a sheet-like implant) to a treatment site of the patient P may require a physician to create one or more incisions (e.g., portals) in the patient P sufficient to access the treatment site. The physician may then insert an implant delivery device through the one or more incisions (e.g., portals) and position a distal end of the implant delivery device adjacent the treatment site. The physician may then manipulate the implant delivery device to deploy a medical implant (e.g., the sheet-like implant) out of a delivery sheath at and/or adjacent the treatment site. As such, FIG. 1 illustrates the one or more incisions (e.g., portals) formed through a patient's skin to access the treatment site at a joint region (e.g., the shoulder 10) of the patient P. In some embodiments, the one or more incisions (e.g., portals) may include a lateral portal 11 and/or a superior portal 13. Other configurations are also contemplated.

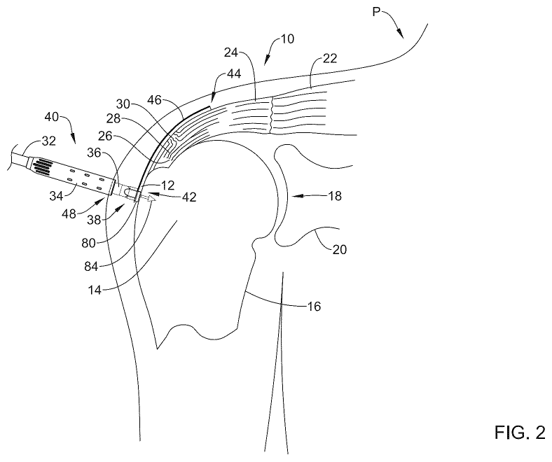

FIG. 2 shows a cross-sectional view of the shoulder 10 with a sheet-like implant 12 being deployed onto soft tissue (e.g., tendon, muscle, etc.) at the treatment site at the joint region (e.g., the shoulder 10) of the patient P. The shoulder 10 includes a head 14 of the humerus 16 mating with a glenoid fossa 18 of the scapula 20. The glenoid fossa 18 comprises a shallow depression in the scapula 20. A supraspinatus tendon 22 is also shown. A distal tendon 24 of the supraspinatus tendon 22 meets the humerus 16 at an insertion point 26.

In FIG. 2, the tendon 24 includes a damaged portion 28 located near the insertion point 26. The damaged portion 28 includes a tear 30 extending partially through the tendon 24. The tear 30 may be referred to as a partial thickness tear. The partial thickness tear 30 is shown on the bursal side of the tendon, however, the tear may also be on the opposite or articular side of the tendon 24 and/or may include internal tears to the tendon 24 not visible on or from either surface or side of the tendon 24.

In FIG. 2, an implant delivery device 40 is shown extending through the skin of the shoulder 10 of the patient P and deployed adjacent the treatment site (e.g., the tear 30 in the tendon 24). In at least some embodiments, the implant delivery device 40 may include an outer shaft 32 (e.g., a cannula) including a proximal end (not shown), a distal end, and a lumen extending within at least a portion of the outer shaft 32. In some embodiments, the distal end of the outer shaft 32 may be attached to a delivery sheath 34. As such, the delivery sheath 34 may extend away from the distal end of the outer shaft 32 whereby the distal end of the outer shaft 32 may be attached to a proximal end of the delivery sheath 34. In some embodiments, the delivery sheath 34 may be and/or may look like a substantially cylindrical sheath, a portion of which may be over-molded onto the distal end of the outer shaft 32. The delivery sheath 34 may be designed to house the sheet-like implant 12 in a rolled state as the implant delivery device 40 is advanced to the treatment site.

FIG. 2 further illustrates that the implant delivery device 40 may include an inner shaft 36 extending within the lumen of the outer shaft 32 and longitudinally movable relative thereto. The inner shaft 36 may include a proximal end (not shown) extending out of the proximal end of the outer shaft 32 and/or otherwise manipulatable relative to the outer shaft 32 by a user. Additionally, in some embodiments, the proximal end of the inner shaft 36 and/or the outer shaft 32 may be coupled to a handle member (not shown). The handle member may be utilized to manipulate the inner shaft 36 relative to the outer shaft 32 and delivery sheath 34. In some embodiments, the handle member may be utilized to impart a rotational force to the inner shaft 36 and/or longitudinal movement of the inner shaft 36 relative to the outer shaft 32 and delivery sheath 34.

The inner shaft 36 may include a distal end region 38, which is shown extending out of the distal end of the delivery sheath 34 in FIG. 2. The inner shaft 36 may include a lumen extending therein. The lumen of the inner shaft 36 may extend along a portion or the entire length of the inner shaft 36 (e.g., from the distal end region 38 to the proximal end of the inner shaft 36, or some portion thereof).

The implant delivery device 40 may further include a frame 46 attached to the distal end region 38 of the inner shaft 36. In some embodiments, the frame 46 may be detachable from the inner shaft 36 in vivo. As shown in FIG. 2, the sheet-like implant 12 may be releasably attached to the frame 46 for delivery to and deployment at the treatment site. For purposes of the discussion herein, a combined structure including the frame 46 and the sheet-like implant 12 may be defined as having a proximal end 42 and a distal end 44 as illustrated in FIG. 2.

When initially positioning the frame 46 and the sheet-like implant 12 adjacent a treatment site, the user may orient the frame 46 and the sheet-like implant 12 (for example, via the handle member attached to the proximal end of the inner shaft 36) such that the proximal end 42 of the combined structure (e.g., the frame 46 and the sheet-like implant 12) may be adjacent (e.g., overlaid) on a portion of the head 14 of the humerus 16 (e.g., on the bone), while the distal end 44 of the combined structure (e.g., the frame 46 and the sheet-like implant 12) may overlay the tendon 24.

In some embodiments, the method of treating soft tissue may include inserting the implant delivery device 40 (e.g., the outer shaft 32 and the delivery sheath 34) through the lateral portal 11 (e.g., FIG. 1) and advancing the implant delivery device 40 to the treatment site with the frame 46 and the sheet-like implant 12 contained within a distal portion of the lumen of the delivery sheath 34. After positioning the distal end 48 of the delivery sheath 34 proximate the treatment site, the method may include deploying the sheet-like implant 12 from the implant delivery device 40 onto the soft tissue at the treatment site. In some embodiments, deploying the sheet-like implant 12 from the implant delivery device 40 may include deploying the combined structure (e.g., the frame 46 and the sheet-like implant 12) out of the lumen of the delivery sheath 34, such as by retracting the outer shaft 32 and the delivery sheath 34 relative to the inner shaft 36. The combined structure (e.g., the frame 46 and the sheet-like implant 12) may automatically expand to a deployed configuration when unconstrained by the delivery sheath 34. In some embodiments, the frame 46 may be “shape set” such that the deployed configuration may generally match the curvature of the head 14 of the humerus 16. As such, the frame 46 may expand to a substantially curved configuration which matches the curvature of the head 14 of the humerus 16 when unconstrained by the delivery sheath 34. In some embodiments, the frame 46 may be biased toward the deployed configuration and/or the substantially curved configuration when unconstrained. In some embodiments, the frame 46 may be self-biased toward the deployed configuration and/or the substantially curved configuration when unconstrained. The method may subsequently include positioning the combined structure (e.g., the frame 46 and the sheet-like implant 12) over the treatment site and/or onto the soft tissue.

As discussed above, prior to deployment, the frame 46 and the sheet-like implant 12 may be contained (e.g., housed) within the lumen of the delivery sheath 34 for subsequent deployment distally out the distal opening of the delivery sheath 34. In some embodiments, the combined structure (e.g., the frame 46 and the sheet-like implant 12) may wrap and/or fold upon itself such that it may be positioned within the lumen of the delivery sheath 34. Alternatively, in some embodiments, the combined structure (e.g., the frame 46 and the sheet-like implant 12) may wrap and/or fold around the inner shaft 36 while disposed within the delivery sheath 34.

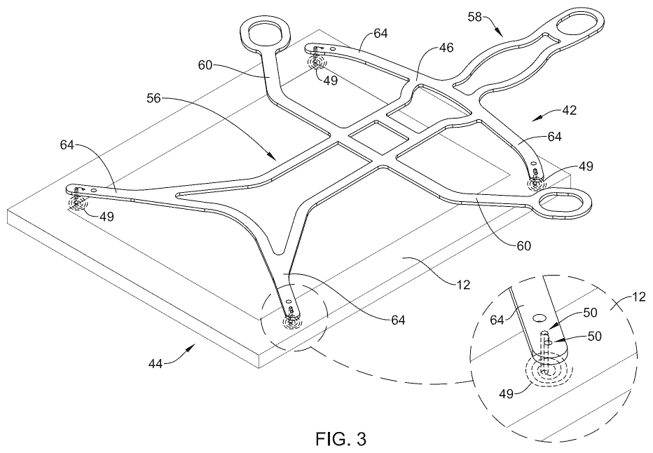

FIG. 3 shows selected aspects of the frame 46 attached to the sheet-like implant 12. As stated above with reference to FIG. 2, the frame 46 and the sheet-like implant 12 may have a proximal end 42 which, for purposes of discussion herein, may be adjacent the connection to the inner shaft 36 and be configured to be positioned adjacent the head 14 of the humerus 16. Further, the frame 46 and the sheet-like implant 12 may have a distal end 44 which, for purposes of discussion herein, may extend away from the inner shaft 36 and may be configured to be positioned adjacent to the tendon 24.

As shown in FIG. 3, the frame 46 may include a body portion 56 and a head portion 58. The body portion 56 may include a plurality of attachment arms 64 extending laterally therefrom. The body portion 56 may include one or more connector legs 60 extending laterally therefrom. The head portion 58 and/or the one or more connector legs 60 may be configured to attach the frame 46 to a portion of the implant delivery device 40. In at least some embodiments, the head portion 58 and/or the one or more connector legs 60 may be configured to attach the frame 46 to the inner shaft 36 and/or the distal end region 38 of the inner shaft 36.

In some embodiments, the frame 46 may include one or more coil attachment members 49 which may be utilized to releasably couple the frame 46 to the sheet-like implant 12. In some embodiments, the frame 46 and/or the one or more coil attachment members 49 may include four coil attachment members, however, it is contemplated that more (or less) than four attachment members may be utilized to releasably couple the frame 46 to the sheet-like implant 12. In some embodiments, a first portion of each of the one or more coil attachment members 49 may be threaded through one or more attachment apertures 50 located on and/or formed in the plurality of attachment arms 64 of the frame 46 while a second portion of each of the one or more coil attachment members 49 may be coiled on a bottom side of the sheet-like implant 12, whereby the sheet-like implant 12 is sandwiched between the second portion of each of the one or more coil attachment members 49 and the frame 46.

Returning briefly to FIG. 2, the inner shaft 36 and/or the distal end region 38 of the inner shaft 36 may be coupled to the frame 46 via a connection assembly. The connection assembly may include a collar (not shown) and a tack disk 80. In at least some embodiments, the connection assembly may be coupled to the head portion 58 and the one or more connector legs 60. In some embodiments, the head portion 58 and the one or more connector legs 60 may be sandwiched and/or constrained between the collar and the tack disk 80. In some embodiments, the implant delivery device 40 may include a tack member 84 extending distally from the tack disk 80 and/or the frame 46. In some embodiments, the tack member 84 may be oriented substantially perpendicular to the sheet-like implant 12 and/or the frame 46. In some embodiments, the tack member 84 may extend generally parallel to a longitudinal axis of the outer shaft 32 and/or the inner shaft 36 with the frame 46 and the sheet-like implant 12 extending generally perpendicular to the longitudinal axis of outer shaft 32 and/or the inner shaft 36. However, this configuration is not intended to be limiting. In some embodiments, the tack member 84 may extend distally from the tack disk 80 and/or the frame 46 at an oblique angle to the longitudinal axis of the outer shaft 32, the inner shaft 36, and/or the frame 46.

In some embodiments, the tack member 84 may resemble a cylindrical pin or rod extending distally away from the tack disk 80 and/or the frame 46. In some embodiments, the tack member 84 may be designed to be rigid enough to be pounded and/or inserted into bone. In some embodiments, the user may apply a force to a proximal portion of the implant delivery device 40 (e.g., the inner shaft 36) such that the tack member 84 may be driven into a body structure (e.g., bone). In some embodiments, the tack member 84 may include a tapered distal tip, which may be a sharpened or blunt tapered distal tip. In some embodiments, the tack member 84 may be stationary (e.g., fixed in place) relative to the frame 46, the collar, and/or the tack disk 80. For example, the tack member 84 may extend distally from the tack disk 80 and distally away from a distal surface of the frame 46 which faces the treatment site. In some embodiments, the tack member 84 may extend through an aperture defined in the body portion 56 when the frame 46 is in the deployed configuration. The aperture may be sized such that the distal tip of the tack member 84 may be prevented from passing proximally out through the aperture when the frame 46 is flexed relative to the inner shaft 36, thus maintaining the distal tip of the tack member 84 on a distal side of the frame 46 for engagement with a bone during implantation of the sheet-like implant 12. Furthermore, in some embodiments, the tack member 84 may extend through the sheet-like implant 12 when attached to the frame 46 in the deployed configuration. In FIG. 2, the tack member 84 is shown extending through the sheet-like implant 12 and into the head 14 of the humerus 16.

In some embodiments, the tack member 84 may be configured to secure the sheet-like implant 12 to soft tissue and/or bone (e.g., to the head 14 of the humerus 16) to hold the sheet-like implant 12 in place while securing the sheet-like implant 12 to the soft tissue and/or bone adjacent the treatment site using at least one bone anchor and/or at least some soft tissue anchors. In some embodiments, the tack member 84 may be detachable from the tack disk 80 and/or the inner shaft 36. In some embodiments, after deploying the tack member 84 into the sheet-like implant 12 and/or bone (e.g., the head 14 of the humerus 16) adjacent the treatment site, the tack member 84 and the frame 46 may be detached from the implant delivery device 40 and the implant delivery device 40 may be removed from the treatment site and/or the lateral portal 11. Use of the tack member 84 may permit the implant delivery device 40 to be removed from the treatment site and/or the lateral portal 11 while other devices are inserted and/or disposed within the lateral portal 11, thus reducing overall size of the lateral portal 11 and/or eliminating or reducing the need for additional portals, which may improve healing and/or reduce scarring of the skin after treatment, along with other benefits. After at least one bone anchor and/or at least some soft tissue anchors have been deployed at the treatment site and/or the sheet-like implant 12 has been secured to the soft tissue and/or bone at the treatment site, the tack member 84 and the frame 46 may be retrieved from the treatment site. Subsequently, the lateral portal 11 (and/or any other portals) may be closed and/or sealed (e.g., sutured, stapled, adhered closed, etc.).

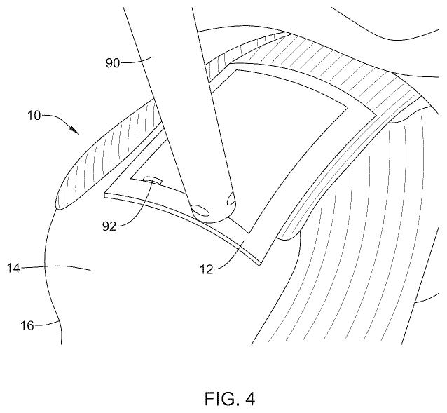

In some embodiments, a method of treating soft tissue at the joint region (e.g., the shoulder 10) of the patient P may include inserting a bone anchor delivery device 90, shown in FIG. 4, through the lateral portal 11 to the treatment site. The method may include deploying at least one bone anchor 92 from the bone anchor delivery device 90 to secure the sheet-like implant 12 to bone (e.g., the head 14 of the humerus 16) adjacent the treatment site and/or the tendon 24. In some embodiments, the at least one bone anchor 92 may include two bone anchors, three bone anchors, four bone anchors, etc., depending on how much of the sheet-like implant 12 overlays bone instead of soft tissue. Other configurations are also contemplated. As will be discussed in more detail herein, in some embodiments, the method may include deploying at least one soft tissue anchor into the sheet-like implant 12 and the soft tissue at the treatment site. In some embodiments, the at least one soft tissue anchor may include a first soft tissue anchor, a second soft tissue anchor, etc. In some embodiments, the at least one bone anchor 92 may be deployed prior to deploying the at least one soft issue anchor (e.g., the first soft tissue anchor, etc.). In some embodiments, the at least one bone anchor may be deployed subsequent to deploying the at least one soft tissue anchor (e.g., the first soft tissue anchor, etc.). Other configurations and/or step sequencing are also contemplated.

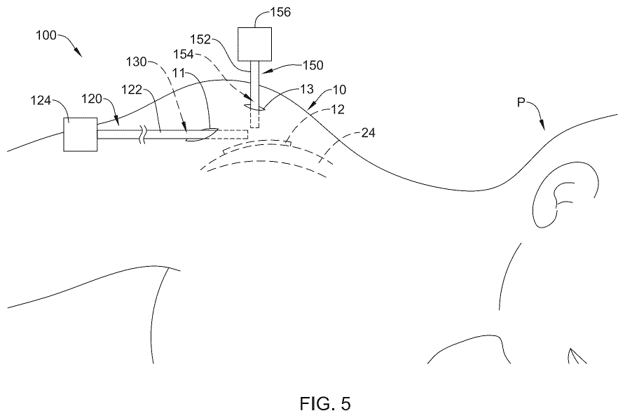

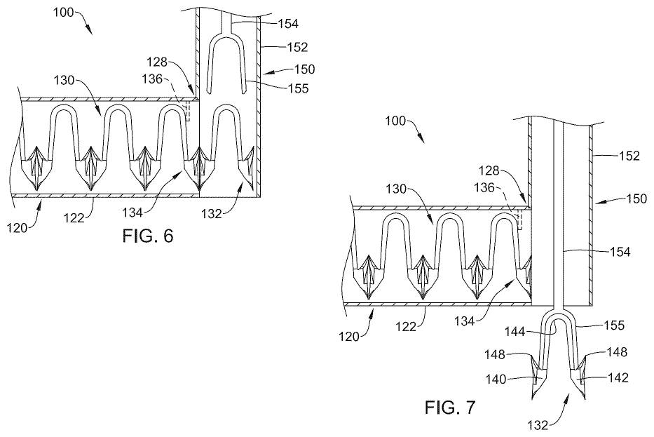

FIG. 5 illustrates selected aspects of a system 100 for securing the sheet-like implant 12 to soft tissue and/or bone at the treatment site, and/or a method of treating soft tissue at a joint region (e.g., the shoulder 10) of the patient P. As discussed herein, the joint region may be the treatment site, and these terms may be used interchangeably herein. The system 100 may include a tissue anchor delivery device 120 including an elongate shaft 122 having a longitudinal axis and a plurality of tissue anchors 130 movably disposed within a lumen of the elongate shaft 122. Additional details related to the plurality of tissue anchors 130 may be seen in and/or are discussed with respect to FIGS. 6-9. In some embodiments, the tissue anchor delivery device 120 may include a handle 124 disposed proximate a proximal end of the elongate shaft 122. In FIG. 5, the handle 124 is shown schematically and the illustrated view is not intended to be limiting in form or function.

The system 100 may include a tissue anchor deployment device 150 separate from the tissue anchor delivery device 120. The tissue anchor deployment device 150 may include an outer tube 152 and an inner member 154 (e.g., FIGS. 6-9) movably disposed within the outer tube 152. In some embodiments, the tissue anchor deployment device 150 may include a handle 156 disposed proximate a proximal end of the outer tube 152. In FIG. 5, the handle 156 is shown schematically and the illustrated view is not intended to be limiting in form or function.

In some embodiments, the tissue anchor deployment device 150 and/or the outer tube 152 may be configured for insertion through a separate incision and/or portal formed in the skin of the patient P than the tissue anchor delivery device 120. In one example, the tissue anchor delivery device 120 may be configured for insertion through the lateral portal 11 to the treatment site and the tissue anchor deployment device 150 and/or the outer tube 152 may be configured for insertion through the superior portal 13 to the treatment site. Other configurations, including but not limited to a reverse configuration, are also contemplated.

In some embodiments, the tissue anchor deployment device 150 may be oriented non-parallel to the elongate shaft 122 (and/or the longitudinal axis of the elongate shaft 122) of the tissue anchor delivery device 120. In some embodiments, the tissue anchor deployment device 150 may be oriented generally perpendicular to the elongate shaft 122 (and/or the longitudinal axis of the elongate shaft 122) of the tissue anchor delivery device 120. The tissue anchor deployment device 150 and/or the outer tube 152 may be configured to engage the elongate shaft 122 of the tissue anchor delivery device 120 proximate a distal end of the elongate shaft 122.

In some embodiments, the plurality of tissue anchors 130 may include a first tissue anchor 132, a second tissue anchor 134, etc., as seen in FIGS. 6-9. In some embodiments, each of the plurality of tissue anchors 130 may be releasably attached to an adjacent one of the plurality of tissue anchors 130. For example, the first tissue anchor 132 may be releasably attached to the second tissue anchor 134, etc. In some embodiments, each of the plurality of tissue anchors 130 may be releasably attached to an adjacent one of the plurality of tissue anchors 130 by a frangible connection. In some embodiments, each of the plurality of tissue anchors 130 may be releasably attached to an adjacent one of the plurality of tissue anchors 130 by an adhesive connection. Other configurations are also contemplated.

In some embodiments, the tissue anchor deployment device 150 and/or the inner member 154 may be configured to engage the first tissue anchor 132 of the plurality of tissue anchors 130 proximate the distal end of the elongate shaft 122 of the tissue anchor delivery device 120 to insert the first tissue anchor 132 of the plurality of tissue anchors 130 into the sheet-like implant 12 and the soft tissue and/or bone at the treatment site to secure the sheet-like implant 12 thereto. In some embodiments, the tissue anchor deployment device 150 and/or the inner member 154 may be configured to subsequently engage the second tissue anchor 134 of the plurality of tissue anchors 130 proximate the distal end of the elongate shaft 122 of the tissue anchor delivery device 120 to insert the second tissue anchor 134 of the plurality of tissue anchors 130 into the sheet-like implant 12 and the soft tissue and/or bone at the treatment site to secure the sheet-like implant 12 thereto. Additional tissue anchors of the plurality of tissue anchors 130 may be deployed in a similar manner as needed or desired.

As shown in FIGS. 6-9, the plurality of tissue anchors 130 each include a first prong 140, a second prong 142 spaced apart from the first prong 140, and a bridge 144 extending from the first prong 140 to the second prong 142. The first prong 140 and the second prong 142 may extend and/or may be oriented transverse to the longitudinal axis of the elongate shaft 122. In some embodiments, the bridge 144 may be generally parallel to and/or oriented in alignment with the longitudinal axis of the elongate shaft 122. In some embodiments, the first prong 140 and/or the second prong 142 may include a barb 148 extending proximally and/or laterally therefrom. The barb 148 may be configured to aid in retaining the first prong 140 and/or the second prong 142 within soft tissue and/or the sheet-like implant 12, by preventing and/or making it harder for the first prong 140 and/or the second prong 142 to be pulled proximally out of the soft tissue and/or the sheet-like implant 12. In some embodiments, each of the plurality of tissue anchors 130 may be releasably attached to an adjacent one of the plurality of tissue anchors 130 in a prong-to-prong arrangement (e.g., the first prong 140 of the first tissue anchor 132 may be engaged with and/or releasably attached to the second prong 142 of the second tissue anchor 134, etc.).

The tissue anchor deployment device 150 and/or the inner member 154 may be configured to engage the bridge 144 (and/or the first prong 140 and the second prong 142) to insert the first prong 140 and/or the second prong 142 into the sheet-like implant 12 and the soft tissue and/or bone. In some embodiments, the inner member 154 may include a distal fork 155 configured to engage the first prong 140, the second prong 142, and/or the bridge 144.

As discussed herein, the tissue anchor deployment device 150 and/or the outer tube 152 may be configured to engage the elongate shaft 122 of the tissue anchor delivery device 120 proximate the distal end of the elongate shaft 122. In some embodiments, a distal end of the outer tube 152 may be configured to engage and/or contact the elongate shaft 122 of the tissue anchor delivery device 120 proximate the distal end of the elongate shaft 122.

In some embodiments, the elongate shaft 122 includes an engagement feature configured to engage a mating engagement feature of the outer tube 152. In some embodiments, the elongate shaft 122 of the tissue anchor delivery device 120 may include and/or the engagement feature may be a notch 128 formed in a wall of the elongate shaft 122 proximate the distal end of the elongate shaft 122, the notch 128 being configured to slidably receive at least a portion of the distal end of the outer tube 152 of the tissue anchor deployment device 150. In some embodiments, the at least a portion of the distal end of the outer tube 152 may be the mating engagement feature. In some embodiments, the notch 128 may extend only partially through the wall of the elongate shaft 122. In some alternative embodiments, the notch 128 may extend completely through the wall of the elongate shaft 122. In some embodiments, the outer tube 152 may include a cutout extending proximally from a distalmost extent of the outer tube 152, wherein the cutout is configured to engage the elongate shaft 122 and/or extend into the notch 128 formed in the wall of the elongate shaft 122. In some embodiments, the cutout may be the mating engagement feature.

Alternatively, in some embodiments, the elongate shaft 122 of the tissue anchor delivery device 120 may include and/or the engagement feature may be a projection (not shown) extending therefrom and the outer tube 152 of the tissue anchor deployment device 150 may include and/or the mating engagement feature may be a notch (not shown) configured to slidably receive the projection therein. In one example, the projection may extend radially from the wall of the elongate shaft 122. In another example, the projection may extend distally from the distal end of the elongate shaft 122. In another alternative embodiment, the outer tube 152 may include and/or the mating engagement feature may be a projection extending therefrom and configured to extend into the notch 128, wherein the notch 128 is configured to slidably receive the projection therein. Other configurations are also contemplated.

In some embodiments, the tissue anchor delivery device 120 may include a biasing mechanism (not shown) configured to advance the plurality of tissue anchors 130 distally within the elongate shaft 122. In some embodiments, the biasing mechanism may be configured to automatically advance the plurality of tissue anchors 130 distally within the elongate shaft 122 when the distalmost one of the plurality of tissue anchors 130 is deployed. In some embodiments, the biasing mechanism may be configured to manually advance the plurality of tissue anchors 130 distally within the elongate shaft 122 after the distalmost one of the plurality of tissue anchors 130 is deployed. In one example, the biasing mechanism may include a coil spring disposed within the tissue anchor delivery device 120 and/or the elongate shaft 122. Other biasing mechanisms are also contemplated—including but not limited to ratcheting mechanisms, push rods, etc.

In some embodiments, the elongate shaft 122 of the tissue anchor delivery device 120 may include a stop feature 136 proximate and/or at the distal end of the elongate shaft 122. The stop feature 136 may be configured to prevent distal advancement of the distalmost one of the plurality of tissue anchors 130 past a predetermined point. In at least some embodiments, the first tissue anchor 132 and/or the distalmost tissue anchor of the plurality of tissue anchors 130 may be exposed to an exterior of the elongate shaft 122 proximate the distal end of the elongate shaft 122.

In some embodiments, the first tissue anchor 132 and/or the distalmost tissue anchor of the plurality of tissue anchors 130 may be disposed distal of the distal end of the elongate shaft 122, as shown in FIGS. 6-7. In some embodiments, when the outer tube 152 is engaged with the elongate shaft 122, a majority of the outer tube 152 is disposed distal of the distal end of the elongate shaft 122. In the configuration illustrated in FIGS. 6-7, each of the plurality of tissue anchors 130 has a length measured axially along the longitudinal axis of the elongate shaft 122 and the predetermined point is disposed distal of the distal end of the elongate shaft 122 between 100% and 125% of the length. To accomplish this, the stop feature 136 may be movable, retractable, pivotable, slidable, etc. between a first position and a second position. The stop feature 136 may be movable proximally, distally, radially, transversely, etc. relative to the longitudinal axis of the elongate shaft 122.

The stop feature 136 may extend radially inward from the wall of the elongate shaft 122 proximate the distal end of the elongate shaft 122 in the first position. In some embodiments, the stop feature 136 may be at the distal end of the elongate shaft 122. In some embodiments, the stop feature 136 may be disposed proximal of the distal end of the elongate shaft 122. The stop feature 136 may be movable, retractable, pivotable, slidable, etc. from the first position to the second position to permit the plurality of tissue anchors 130 to advance distally. In some embodiments, the stop feature 136 may be configured to automatically move, extend, pivot, slide, etc. from the second position into and/or across the lumen of the elongate shaft 122 to the first position to at least partially obstruct the lumen and prevent distal advancement of the distalmost one (e.g., the first tissue anchor 132 in FIG. 6) of the plurality of tissue anchors 130 past the predetermined point.

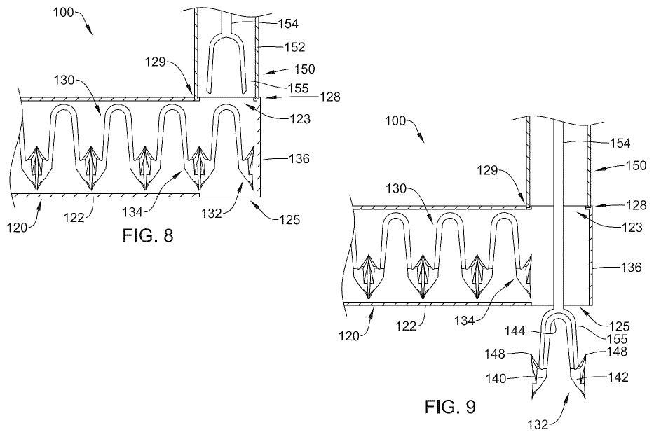

When the outer tube 152 is engaged with the elongate shaft 122, the inner member 154 and/or the distal fork 155 may be configured to detach the first tissue anchor 132 and/or the distalmost tissue anchor of the plurality of tissue anchors 130 from second tissue anchor 134 and/or the adjacent tissue anchor of the plurality of tissue anchors 130 and deploy the first tissue anchor 132 and/or the distalmost tissue anchor of the plurality of tissue anchors 130 into the sheet-like implant 12 and the soft tissue and/or bone at the treatment site upon actuation and/or distal advancement of the inner member 154 relative to the outer tube 152. In some embodiments, the elongate shaft 122 may include a side facing window 123 through the wall of the elongate shaft 122, as seen in FIGS. 8-9. In at least some embodiments, the side facing window 123 may be disposed proximal of the distal end of the elongate shaft 122. In some embodiments, the elongate shaft 122 may include a second side facing window 125 disposed opposite the side facing window 123 relative to the longitudinal axis of the elongate shaft 122. In some embodiments, the elongate shaft 122 may include a second engagement feature (e.g., a second notch 129) disposed proximal of the engagement feature (e.g., the notch 128). The engagement feature (e.g., the notch 128) and the second engagement feature (e.g., the second notch 129) may be configured to engage and/or receive at least a portion of the outer tube 152.

In some embodiments, when the outer tube 152 is engaged with the elongate shaft 122, the inner member 154 and/or the distal fork 155 may be configured to engage the first tissue anchor 132 and/or the distalmost tissue anchor of the plurality of tissue anchors 130 through the side facing window 123. When the outer tube 152 is engaged with the elongate shaft 122, the inner member 154 and/or the distal fork 155 may be configured to eject the first tissue anchor 132 and/or the distalmost tissue anchor of the plurality of tissue anchors 130 through the second side facing window 125 and deploy the first tissue anchor 132 and/or the distalmost tissue anchor of the plurality of tissue anchors 130 into the sheet-like implant 12 and the soft tissue and/or bone at the treatment site upon actuation and/or distal advancement of the inner member 154 relative to the outer tube 152. In some embodiments, the stop feature 136 may be fixed and/or immovable relative to a wall of the elongate shaft 122. In some embodiments, the stop feature 136 may be the distal end of the elongate shaft 122, which may be a closed distal end, as shown in FIGS. 8-9. As such, the predetermined point may be the distal end of the elongate shaft 122. Other configurations are also contemplated.

In some alternative configurations to those shown in FIGS. 6-9, the plurality of tissue anchors 130 may be oriented perpendicular to the orientation shown in the figures. For example, in some embodiments, the bridge 144 may extend and/or may be oriented generally transverse to and/or perpendicular to the longitudinal axis of the elongate shaft 122. In some embodiments, each of the plurality of tissue anchors 130 may be releasably attached to an adjacent one of the plurality of tissue anchors 130 in a prong-to-prong arrangement (e.g., the first prong 140 of the first tissue anchor 132 may be engaged with and/or releasably attached to the second prong 142 of the second tissue anchor 134, etc.). Alternatively, in some embodiments, each of the plurality of tissue anchors 130 may be releasably attached to an adjacent one of the plurality of tissue anchors 130 in a bridge-to-bridge arrangement (e.g., the bridge 144 of the first tissue anchor 132 may be engaged with and/or releasably attached to the bridge 144 of the second tissue anchor 134, etc.). In some further alternative embodiments, each of the plurality of tissue anchors 130 may be releasably attached to an adjacent one of the plurality of tissue anchors 130 in both a prong-to-prong and a bridge-to-bridge arrangement.

A method of treating soft tissue at the joint region (e.g., the shoulder 10) of the patient P may include creating the lateral portal 11 and the superior portal 13 through the patient's skin to access the treatment site at the joint region, as seen in FIG. 1. The method may include inserting the implant delivery device 40 through the lateral portal 11 and positioning the distal end of the implant delivery device 40 over the soft tissue (e.g., the tendon 24) and/or at the treatment site, as seen in FIG. 2. The method may include deploying the sheet-like implant 12 from the implant delivery device 40 onto the soft tissue (e.g., the tendon 24) at the treatment site. In some embodiments, the method may include securing the sheet-like implant 12 in place over the soft tissue (e.g., tendon 24) and/or at the treatment site with the tack member 84. In some embodiments, the method may include removing the implant delivery device 40 from the lateral portal 11 and/or the patient P while leaving and/or maintaining the sheet-like implant 12 in position over the soft tissue (e.g., the tendon 24) and/or at the treatment site.

The method may include inserting the tissue anchor delivery device 120 into and/or through the lateral portal 11 to the treatment site, as seen in FIG. 5. In at least some embodiments, inserting the tissue anchor delivery device 120 into and/or through the lateral portal 11 to the treatment site may occur after removing the implant delivery device 40 from the lateral portal 11 and/or the patient P. The method may also include inserting the tissue anchor deployment device 150 non-parallel to the longitudinal axis of the elongate shaft 122 into and/or through the superior portal 13 to the treatment site, as also shown in FIG. 5. In some embodiments, the use of two separate devices for delivering and/or deploying the plurality of tissue anchors 130 may permit smaller devices to be used, thereby reducing the size of the portal(s) necessary to perform the treatment.

In some embodiments, the method may include engaging the tissue anchor deployment device 150 and/or the outer tube 152 with the tissue anchor delivery device 120 and/or the elongate shaft 122. In some embodiments, the method may include engaging the engagement feature of the elongate shaft 122 with the mating engagement feature of the outer tube 152. In some embodiments, the method may include engaging the distal end of the outer tube 152 with the notch 128 and/or the second notch 129 formed in the elongate shaft 122. In some embodiments, the method may include engaging the projection extending from the elongate shaft 122 with the notch formed in the outer tube 152. In some embodiments, the method may include engaging the projection extending from the outer tube 152 with the notch 128 formed in the elongate shaft 122. Other configurations are also contemplated.

The method may include engaging the tissue anchor deployment device 150 and/or the distal fork 155 of the inner member 154 with the first tissue anchor 132 of the plurality of tissue anchors 130 and/or with the distalmost one of the plurality of tissue anchors 130 proximate the distal end of the elongate shaft 122. The method may then include deploying the first tissue anchor 132 of the plurality of tissue anchors 130 and/or with the distalmost one of the plurality of tissue anchors 130 into the sheet-like implant 12 to secure the sheet-like implant 12 to the soft tissue (e.g., the tendon 24) and/or bone at the treatment site using the tissue anchor deployment device 150 and/or the inner member 154.

In some embodiments, the method may include, after deploying the first tissue anchor 132 of the plurality of tissue anchors 130 and/or with the distalmost one of the plurality of tissue anchors 130, advancing the remaining tissue anchors of the plurality of tissue anchors 130 distally within the elongate shaft 122 to position the second tissue anchor 134 of the plurality of tissue anchors 130 and/or the distalmost tissue anchor of the remaining tissue anchors of the plurality of tissue anchors 130 proximate the distal end of the elongate shaft 122 and/or to the predetermined point. The method may include engaging the tissue anchor deployment device 150 and/or the distal fork 155 of the inner member 154 with the second tissue anchor 134 of the plurality of tissue anchors 130 and/or with the distalmost tissue anchor of the remaining tissue anchors of the plurality of tissue anchors 130 proximate the distal end of the elongate shaft 122. The method may then include deploying the second tissue anchor 134 of the plurality of tissue anchors 130 and/or with the distalmost tissue anchor of the remaining tissue anchors of the plurality of tissue anchors 130 into the sheet-like implant 12 to secure the sheet-like implant 12 to the soft tissue (e.g., the tendon 24) and/or bone at the treatment site using the tissue anchor deployment device 150 and/or the inner member 154. These steps may be repeated as necessary until all desired tissue anchors of the plurality of tissue anchors 130 have been deployed and/or until the sheet-like implant 12 is sufficiently secured to the soft tissue (e.g., the tendon 24) and/or bone at the treatment site.

In some embodiments, the method may include inserting the bone anchor delivery device 90 into and/or through the lateral portal 11 to the treatment site. In some embodiments, the method may include deploying at least one bone anchor 92 from the bone anchor delivery device 90 into the sheet-like implant 12 to secure the sheet-like implant 12 to soft tissue and/or bone adjacent the treatment site. In some embodiments, the at least one bone anchor 92 may be deployed prior to deploying the first tissue anchor 132 of the plurality of tissue anchors 130 and/or the distalmost one of the plurality of tissue anchors 130 into the sheet-like implant 12. In some embodiments, the at least one bone anchor 92 may be deployed subsequent to or after deploying the first tissue anchor 132 of the plurality of tissue anchors 130 and/or the distalmost one of the plurality of tissue anchors 130 into the sheet-like implant 12.

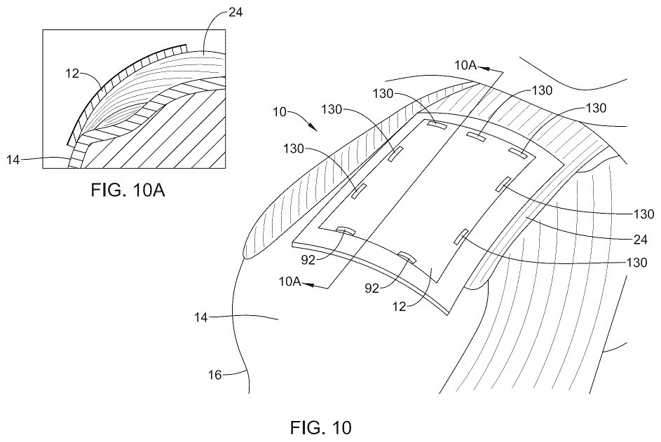

After deploying the at least one bone anchor 92 and/or the plurality of tissue anchors 130 into the sheet-like implant 12 at the treatment site, the shoulder 10 of the patient P may resemble the views shown in FIG. 10 and FIG. 10A.

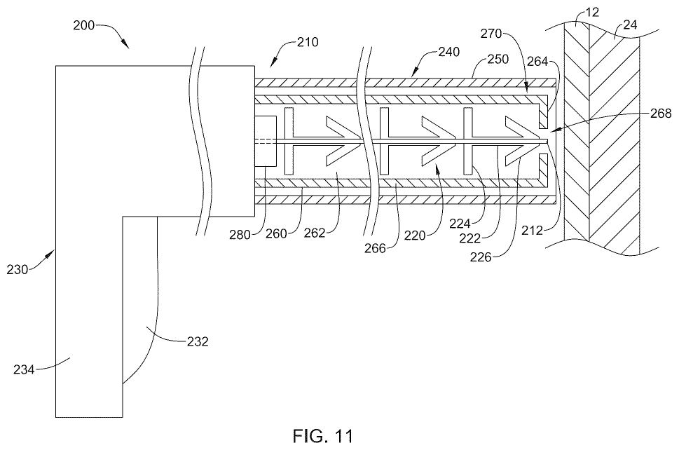

FIGS. 11-18 illustrate selected aspects of a system 200 for securing the sheet-like implant 12 to tissue at the treatment site, and/or a method of treating soft tissue at a joint region (e.g., the shoulder 10) of the patient P. As discussed herein, the joint region may be the treatment site, and these terms may be used interchangeably herein. The system 200 may include a tissue anchor delivery device 210 and a plurality of tissue anchors 220, as shown in FIGS. 11-14. In some embodiments, the tissue anchor delivery device 210 may include a handle 230 and an elongate shaft 240 extending distally from the handle 230 along a longitudinal axis. In some embodiments, the handle 230 may include a trigger member 232 proximate and/or adjacent to a hand grip 234. In FIGS. 11-14, the handle 230 is illustrated schematically and the illustrated views are not intended to be limiting.

The elongate shaft 240 may include an outer sleeve 250 having a lumen extending therein and a tubular deployment member 260 slidably disposed within the lumen of the outer sleeve 250. The tubular deployment member 260 may include a lumen 262 extending therein. In some embodiments, the plurality of tissue anchors 220 may be movably disposed within the lumen 262 of the tubular deployment member 260. In some embodiments, the plurality of tissue anchors 220 may be axially and/or longitudinally movable and/or slidable within the lumen 262 of the tubular deployment member 260.

In some embodiments, each tissue anchor of the plurality of tissue anchors 220 may include a stem 222 extending longitudinally along the longitudinal axis, an enlarged head 224 disposed at a proximal end of the stem 222, and one or more anchor barbs 226 extending laterally from a distal portion of the stem 222. In some embodiments, the enlarged head 224 may extend substantially perpendicular to the stem 222 and/or the longitudinal axis. In some embodiments, the enlarged head 224 may be substantially planar. In some embodiments, the one or more anchor barbs 226 may be oriented at an oblique angle to the stem 222. In some embodiments, the one or more anchor barbs 226 may extend from the stem 222 toward the enlarged head 224. In some embodiments, the one or more anchor barbs 226 may extend radially outward and proximally from the distal portion of the stem 222.

In some embodiments, the plurality of tissue anchors 220 may include a first tissue anchor, a second tissue anchor, etc. In some embodiments, each of the plurality of tissue anchors 220 may be releasably attached to an adjacent one of the plurality of tissue anchors 220. For example, the first tissue anchor may be releasably attached to the second tissue anchor, etc. In some embodiments, each of the plurality of tissue anchors 220 may be releasably attached to an adjacent one of the plurality of tissue anchors 220 by a frangible connection. In some embodiments, each of the plurality of tissue anchors 220 may be releasably attached to an adjacent one of the plurality of tissue anchors 220 by an adhesive connection. Other configurations are also contemplated.

In some embodiments, the tissue anchor delivery device 210 may include a shaft 212 extending longitudinally within and/or through the lumen 262 of the tubular deployment member 260. In at least some embodiments, each of the plurality of tissue anchors 220 may include a central bore extending longitudinally within the stem 222. The shaft 212 may extend through the central bore of each of the plurality of tissue anchors 220 to guide and/or orient the plurality of tissue anchors 220 as the plurality of tissue anchors 220 are advanced within the lumen 262 of the tubular deployment member 260 and/or deployed at the treatment site.

In some embodiments, a distal portion of the tubular deployment member 260 may include at least one flange 264 extending radially inward from a wall 266 of the tubular deployment member 260 to define a distal aperture 268 of the tubular deployment member 260. In at least some embodiments, when the distal portion of the tubular deployment member 260 is disposed within the lumen of the outer sleeve 250, the distal aperture 268 may have a minimum lateral extent measured transverse to the longitudinal axis that is less than a maximum lateral extent of the enlarged head 224 measured transverse to the longitudinal axis.

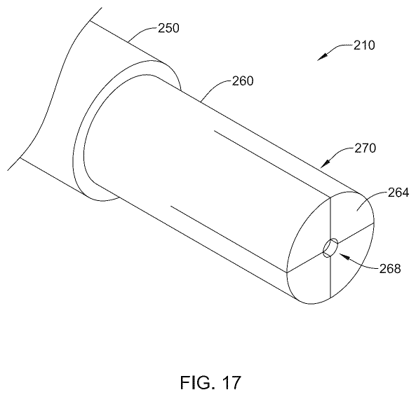

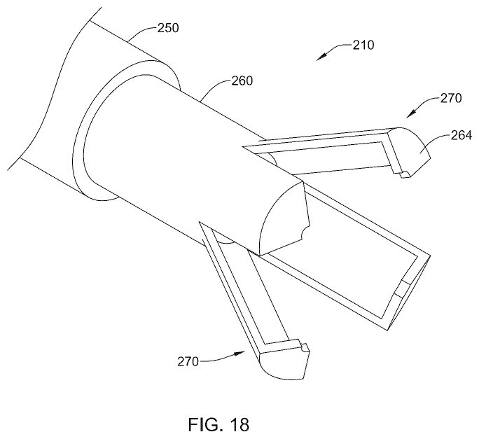

In some embodiments, the distal portion of the tubular deployment member 260 may include a plurality of deflectable strips 270, as seen in FIGS. 17-18. The plurality of deflectable strips 270 may extend proximally from a distal end of the tubular deployment member 260. The plurality of deflectable strips 270 may be formed from the distal portion of the tubular deployment member 260 and/or from the wall 266 of the tubular deployment member 260. While illustrated in FIGS. 17-18 with four deflectable strips, in some embodiments, the plurality of deflectable strips 270 may include two deflectable strips, three deflectable strips, five deflectable strips, six deflectable strips, etc. In some embodiments, the plurality of deflectable strips 270 may include the at least one flange 264. In at least one example, each deflectable strip of the plurality of deflectable strips 270 may include at least one flange 264 extending radially inward therefrom at the distal end of the tubular deployment member 260.

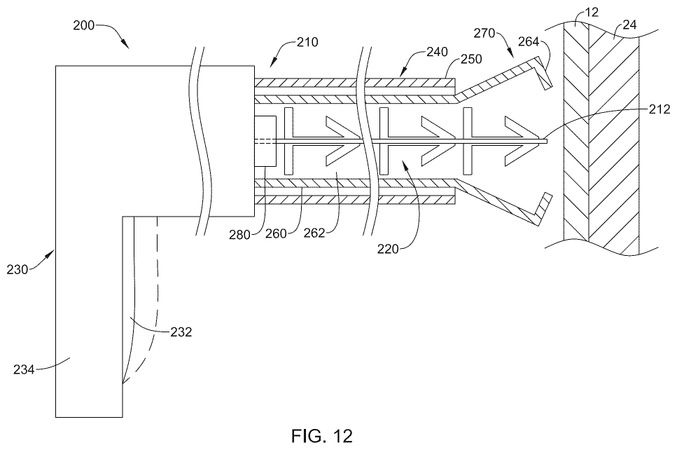

In some embodiments, the plurality of deflectable strips 270 may be movable between a first position (e.g., FIG. 17) and a second position (e.g., FIG. 18). In some embodiments, the plurality of deflectable strips 270 may be movable radially between the first position and the second position. In some embodiments, the first position may be radially closer to the longitudinal axis than the second position. In some embodiments, when the distal portion of the tubular deployment member 260 is disposed within the lumen of the outer sleeve 250, the plurality of deflectable strips 270 are disposed in the first position, as seen in FIG. 11. In at least some embodiments, the plurality of deflectable strips 270 are biased radially outwardly toward the second position when the distal portion of the tubular deployment member 260 is unconstrained by the outer sleeve 250, as seen in FIG. 12. In some embodiments, the plurality of deflectable strips 270 may be self-biased radially outwardly toward the second position when the distal portion of the tubular deployment member 260 is unconstrained by the outer sleeve 250. In some embodiments, the plurality of deflectable strips 270 may be actuatable between the first position and the second position. In some embodiments, the plurality of deflectable strips 270 may be manually actuatable between the first position and the second position. Other configurations are also contemplated.

In some embodiments, actuation of the trigger member 232 on the handle 230 may translate the outer sleeve 252 relative to the tubular deployment member 260, as seen in FIG. 12. In some embodiments, actuation of the trigger member 232 on the handle 230 may translate the outer sleeve 252 proximally relative to the tubular deployment member 260 to expose the plurality of deflectable strips 270 and/or release the distal portion of the tubular deployment member 260 from constrainment by the outer sleeve 250. In some embodiments, actuation of the trigger member 232 on the handle 230 may translate the tubular deployment member 260 distally relative to the outer sleeve 252 to expose the plurality of deflectable strips 270 and/or release the distal portion of the tubular deployment member 260 from constrainment by the outer sleeve 250. Other configurations and/or combinations thereof are also contemplated.

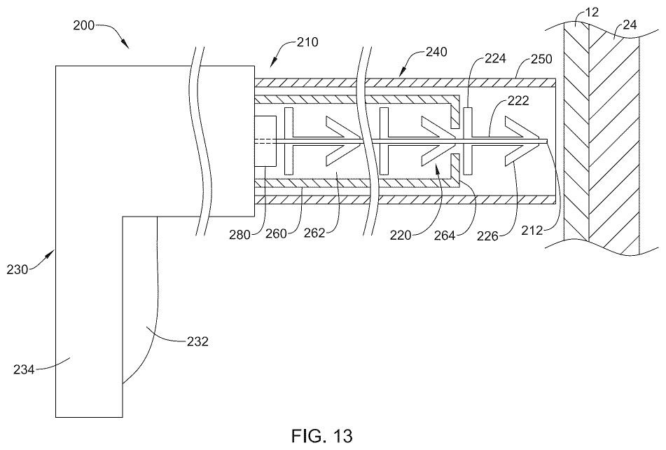

In some embodiments, the plurality of tissue anchors 220 are configured to advance distally relative to the tubular deployment member 260 subsequent to and/or after exposing the plurality of deflectable strips 270 and/or releasing the distal portion of the tubular deployment member 260 from constrainment by the outer sleeve 250, as seen in FIG. 13.

In some alternative embodiments, the plurality of deflectable strips 270 may be biased radially inward toward the first position. In some embodiments, the plurality of deflectable strips 270 may be self-biased radially inward toward the first position. In some embodiments, when the plurality of tissue anchors 220 is advanced distally within the lumen 262 of the tubular deployment member 260 and the distal portion of the tubular deployment member 260 is unconstrained by the outer sleeve 250, a leading one and/or a distalmost one of the plurality of tissue anchors 220 may urge and/or bias the plurality of deflectable strips 270 toward the second position as the leading one and/or the distalmost one of the plurality of tissue anchors 220 passes through the distal aperture 268 and/or moves past the at least one flange 264.

In some embodiments, the tissue anchor delivery device 210 may include a biasing element 280 configured to advance the plurality of tissue anchors 220 distally within the lumen 262 of the tubular deployment member 260 when the distal portion of the tubular deployment member 260 is unconstrained by the outer sleeve 250 and/or when the plurality of deflectable strips 270 are in the second position. In some embodiments, the biasing element 280 may be configured to automatically advance the plurality of tissue anchors 220 distally within the lumen 262 of the tubular deployment member 260 when the distal portion of the tubular deployment member 260 is unconstrained by the outer sleeve 250 and/or when the plurality of deflectable strips 270 are in the second position. In some embodiments, the biasing element 280 may be configured to manually advance the plurality of tissue anchors 220 distally within the lumen 262 of the tubular deployment member 260 when the distal portion of the tubular deployment member 260 is unconstrained by the outer sleeve 250 and/or when the plurality of deflectable strips 270 are in the second position. In one example, the biasing element 280 may include a coil spring disposed within the tissue anchor delivery device 210 and/or the tubular deployment member 260. Other biasing mechanisms are also contemplated—including but not limited to ratcheting mechanisms, push rods, etc.

In some embodiments, after advancing the plurality of tissue anchors 220 distally within the lumen 262 of the tubular deployment member 260, the outer sleeve 250 may be translated distally over and/or relative to the tubular deployment member 260 and/or the plurality of tissue anchors 220 to cover the leading one and/or the distalmost one of the plurality of tissue anchors 220 distal of the tubular deployment member 260, as seen in FIG. 13.

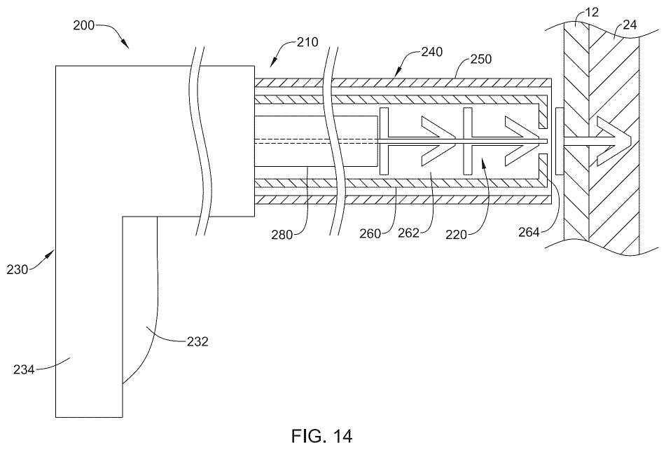

In some embodiments, when the distal portion of the tubular deployment member 260 disposed within and/or is constrained by the outer sleeve 250 and the plurality of deflectable strips 270 are disposed in the first position, and a leading one and/or a distalmost one of the plurality of tissue anchors 220 is disposed distal of the tubular deployment member 260 and/or distal of the at least one flange 264, a distal force applied to the tubular deployment member 260 is configured to insert the leading one and/or the distalmost one of the plurality of tissue anchors 220 into the sheet-like implant 12 and soft tissue and/or bone at the treatment site, as seen in FIG. 14. In at least some embodiments, the outer sleeve 250 may extend distal of the distal end of the tubular deployment member 260 when the distal force is initially applied to the tubular deployment member 260. In some embodiments, application of distal force to the tubular deployment member 260 may translate and/or advance the tubular deployment member 260 distally relative to the outer sleeve 250.

In some embodiments, a distal surface of the distal end of the tubular deployment member 260 (e.g., a distal surface of the at least one flange 264) may be configured to engage the enlarged head 224 of the leading one and/or the distalmost one of the plurality of tissue anchors 220 to insert the leading one and/or the distalmost one of the plurality of tissue anchors 220 into the sheet-like implant 12 and soft tissue and/or bone at the treatment site. In some embodiments, the distal surface of the at least one flange 264 radially overlaps with and/or abuts a proximal surface of the enlarged head 224 of the leading one and/or the distalmost one of the plurality of tissue anchors 220 as the tubular deployment member 260 is translated and/or advanced distally relative to the outer sleeve 250, thereby urging and/or driving the leading one and/or the distalmost one of the plurality of tissue anchors 220 into the sheet-like implant 12 and soft tissue and/or bone at the treatment site. In some embodiments, the stem 222 of the leading one and/or the distalmost one of the plurality of tissue anchors 220 may be oriented generally perpendicular to an upper surface of the sheet-like implant 12 when urging and/or driving the leading one and/or the distalmost one of the plurality of tissue anchors 220 into the sheet-like implant 12 and soft tissue and/or bone at the treatment site.

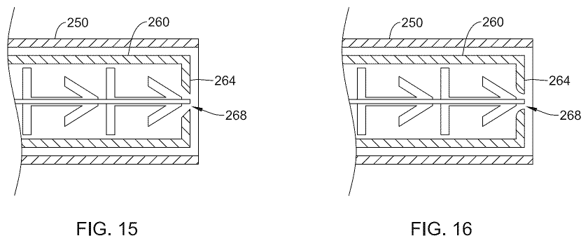

FIGS. 15 and 16 illustrate alternative configurations of the tubular deployment member 260 and/or the at least one flange 264. In some embodiments, the tubular deployment member 260 and/or the at least one flange 264 may include a tapered surface extending distally and radially inward toward the distal aperture 268. In some embodiments, the tapered surface may be a beveled surface, as seen in FIG. 15. In some embodiments, the tapered surface may be a curved surface, as seen in FIG. 16. Other configurations are also contemplated. In some embodiments, the tapered surface may be useful when the plurality of deflectable strips 270 are biased towards the first position. In some embodiments, the tapered surface may cooperate with the one or more anchor barbs 226 to urge and/or bias the plurality of deflectable strips 270 toward the second position.

In some embodiments, less distal force may be needed to translate and/or advance the leading one and/or the distalmost one of the plurality of tissue anchors 220 past the at least one flange 264 when the tapered surface is present. Other benefits are also contemplated.

A method of treating soft tissue at the joint region (e.g., the shoulder 10) of the patient P may include creating the lateral portal 11 and the superior portal 13 through the patient's skin to access the treatment site at the joint region, as seen in FIG. 1. The method may include inserting the implant delivery device 40 through the lateral portal 11 and positioning the distal end of the implant delivery device 40 over the soft tissue (e.g., the tendon 24) and/or at the treatment site, as seen in FIG. 2. The method may include deploying the sheet-like implant 12 from the implant delivery device 40 onto the soft tissue (e.g., the tendon 24) at the treatment site. In some embodiments, the method may include securing the sheet-like implant 12 in place over the soft tissue (e.g., tendon 24) and/or at the treatment site with the tack member 84. In some embodiments, the method may include removing the implant delivery device 40 from the lateral portal 11 and/or the patient P while leaving and/or maintaining the sheet-like implant 12 in position over the soft tissue (e.g., the tendon 24) and/or at the treatment site.

The method may include advancing the tissue anchor delivery device 210 to the treatment site, as seen in FIG. 11. In some embodiments, advancing the tissue anchor delivery device 210 to the treatment site may occur after removing the implant delivery device 40 from the lateral portal 11 and/or the patient P. In some embodiments, the method may include advancing the tissue anchor delivery device 210 to the treatment site through the lateral portal 11. In some embodiments, advancing the tissue anchor delivery device 210 to the treatment site may occur while the implant delivery device 40 is disposed within and/or through the lateral portal 11. In some embodiments, the method may include advancing the tissue anchor delivery device 210 to the treatment site through the superior portal 13.

In some embodiments, the method may include actuating the trigger member 232 on the handle 230 to translate the outer sleeve 250 proximally relative to the tubular deployment member 260 to expose the plurality of deflectable strips 270, as seen in FIG. 12.

In some embodiments, the method may include advancing the plurality of tissue anchors 220 distally relative to the tubular deployment member 260 after exposing the plurality of deflectable strips 270. In some embodiments, after advancing the plurality of tissue anchors 220 distally within the lumen 262 of the tubular deployment member 260, the method may include translating and/or advancing the outer sleeve 250 distally over and/or relative to the tubular deployment member 260 and/or the plurality of tissue anchors 220 to cover the leading one and/or the distalmost one of the plurality of tissue anchors 220 distal of the tubular deployment member 260, as seen in FIG. 13.

The method may include applying a distal force to the tubular deployment member 260 to deploy the leading one and/or the distalmost one of the plurality of tissue anchors 220 into the sheet-like implant 12 and soft tissue and/or bone at the treatment site to secure the sheet-like implant 12 thereto, as seen in FIG. 14. Application of the distal force to the tubular deployment member 260 may translate and/or advancing the tubular deployment member 260 distally relative to the outer sleeve 250 to deploy the leading one and/or the distalmost one of the plurality of tissue anchors 220 into the sheet-like implant 12 and soft tissue and/or bone at the treatment site to secure the sheet-like implant 12 thereto.

In some embodiments, after deploying the leading one and/or the distalmost one of the plurality of tissue anchors 220, these steps may be repeated as necessary until all desired tissue anchors of the plurality of tissue anchors 220 have been deployed and/or until the sheet-like implant 12 is sufficiently secured to the soft tissue (e.g., the tendon 24) and/or bone at the treatment site.

In some embodiments, the method may include inserting the bone anchor delivery device 90 into and/or through the lateral portal 11 to the treatment site. In some embodiments, the method may include deploying at least one bone anchor 92 from the bone anchor delivery device 90 into the sheet-like implant 12 to secure the sheet-like implant 12 to soft tissue and/or bone adjacent the treatment site. In some embodiments, the at least one bone anchor 92 may be deployed prior to deploying the leading one and/or the distalmost one of the plurality of tissue anchors 220 into the sheet-like implant 12. In some embodiments, the at least one bone anchor 92 may be deployed subsequent to or after deploying the leading one and/or the distalmost one of the plurality of tissue anchors 220 into the sheet-like implant 12.

After deploying the at least one bone anchor 92 and/or the plurality of tissue anchors 220 into the sheet-like implant 12 at the treatment site, the shoulder 10 of the patient P may generally resemble the views shown in FIG. 10 and FIG. 10A, except that the plurality of tissue anchors 130 of FIG. 10 would be replaced with the plurality of tissue anchors 220.

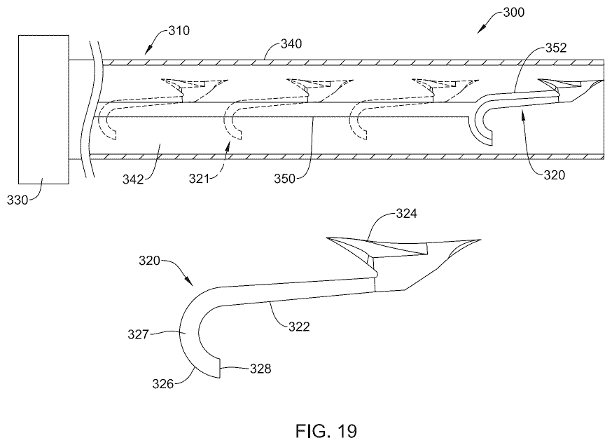

FIG. 19 illustrates selected aspects of a system 300 for securing the sheet-like implant 12 to soft tissue (e.g., the tendon 24) at the treatment site, and/or a method of treating soft tissue at a joint region (e.g., the shoulder 10) of the patient P. As discussed herein, the joint region may be the treatment site, and these terms may be used interchangeably herein.

The system 300 may include a tissue anchor delivery device 310 and a tissue anchor 320. In some embodiments, the system 300 may include at least one tissue anchor and/or a plurality of tissue anchors. In some embodiments, the tissue anchor delivery device 310 may include a handle 330 and a tubular sheath 340 having a lumen 342 extending therein and/or therethrough. The handle 330 may be disposed proximate a proximal end of the tubular sheath 340. In FIG. 19, the handle 330 is shown schematically and the illustrated view is not intended to be limiting in form or function. In some embodiments, the tissue anchor delivery device 310 and/or the tubular sheath 340 may be configured for insertion through the lateral portal 11 to the treatment site. Other configurations are also contemplated.

The tissue anchor delivery device 310 may include an elongate shaft 350 movably disposed within the lumen 342 of the tubular sheath 340 and a distal tine 352 extending distally from the elongate shaft 350. The tissue anchor 320 may be releasably disposable on the distal tine 352. In some embodiments, the distal tine 352 may be configured to pierce the soft tissue (e.g., the tendon 24). In some embodiments, while not expressly shown, the distal tine 352 may protrude distal of at least a portion of the tissue anchor 320. In some embodiments, the distal tine 352 may have a shape and/or form that is complimentary to and/or aligns with at least a portion of the tissue anchor 320.

In some embodiments, the tissue anchor 320 may be movably disposed within the lumen 342 of the tubular sheath 340 on the distal tine 352 prior to delivery and/or deployment of the tissue anchor 320. In some embodiments, the elongate shaft 350 may be engaged with and/or may extend through the handle 330. In some embodiments, the elongate shaft 350 may be configured to be manipulated by the handle 330. In some embodiments, the elongate shaft 350 may be configured to be manipulated by one or more mechanisms of and/or in the handle 330. Other configurations are also contemplated. In some embodiments, the elongate shaft 350 may be fixed to the handle 330 and the tubular sheath 340 may be movable relative to the handle 330 and/or the elongate shaft 350. In some embodiments, the tubular sheath 340 may be fixed to the handle 330 and the elongate shaft 350 may be movable relative to the handle 330 and/or the tubular sheath 340.

FIG. 19 also illustrates selected aspects of the tissue anchor 320 in a standalone view. The tissue anchor is configured for insertion into the sheet-like implant 12 and the soft tissue (e.g., the tendon 24) at the treatment site to secure the sheet-like implant 12 to the soft tissue (e.g., the tendon 24). The tissue anchor 320 may include a single axial prong 322. The tissue anchor 320 may include at least one anchoring barb 324 extending laterally from a distal portion of the single axial prong 322. The tissue anchor 320 may include a tissue securement arm 326 extending laterally from a proximal portion of the single axial prong 322. In some embodiments, the distal tine 352 of the elongate shaft 350 may be configured to engage with the tissue securement arm 326 and/or the single axial prong 322 to insert the tissue anchor 320 into the sheet-like implant 12 and the soft tissue at the treatment site.

In at least some embodiments, the at least one anchoring barb 324 may extend laterally from a first side of the single axial prong 322 and the tissue securement arm 326 may extend laterally from a second side of the single axial prong 322, wherein the second side is opposite the first side relative to the single axial prong. In some embodiments, the at least one anchoring barb 324 may extend laterally from the single axial prong 322 in a first lateral direction and the tissue securement arm 326 may extend from the single axial prong 322 in a second lateral direction opposite the first lateral direction. In some embodiments, the at least one anchoring barb 324 may extend laterally and proximally away from the single axial prong 322. In some embodiments, the at least one anchoring barb 324 may extend laterally and proximally away at an oblique angle from the single axial prong 322. Other configurations are also contemplated. The at least one anchoring barb 324 may be configured to aid in retaining the tissue anchor 320 and/or the single axial prong 322 within soft tissue and/or the sheet-like implant 12, by preventing and/or making it harder for the tissue anchor 320 and/or the single axial prong 322 to be pulled proximally out of the soft tissue and/or the sheet-like implant 12. In some embodiments, the tissue securement arm 326 may include a curved portion 327 extending away from the single axial prong 322 to a free end 328. In at least some embodiments, the free end 328 may extend distal of a proximalmost extent of the curved portion 327 of the tissue securement arm 326.

The tissue anchor 320 may be formed from a biocompatible material. In some embodiments, the tissue anchor 320 may be formed from a resorbable material. In some embodiments, the tissue anchor 320 may be formed from a non-resorbable material. Other configurations, including combinations thereof, are also contemplated.

In some embodiments, the system 300 may optionally include a plurality of tissue anchors 321, illustrated in FIG. 19 in phantom. In at least some embodiments, each of the at least one tissue anchor and/or each of the plurality of tissue anchors 321 may have one or more characteristics of and/or may be the tissue anchor 320. In some embodiments, each of the at least one tissue anchor and/or each of the plurality of tissue anchors 321 may be identical. In some embodiments, one or more of the at least one tissue anchor and/or each of the plurality of tissue anchors 321 may include different forms, shapes, and/or characteristics from the remaining tissue anchors of the plurality of tissue anchors 321. Other configurations are also contemplated.

In some embodiments, each of the at least one tissue anchor and/or each of the plurality of tissue anchors 321 may be releasably attached to an adjacent one of the at least one tissue anchor and/or each of the plurality of tissue anchors 321. For example, a first tissue anchor may be releasably attached to a second tissue anchor, etc. In some embodiments, each of the at least one tissue anchor and/or each of the plurality of tissue anchors 321 may be releasably attached to an adjacent one of the at least one tissue anchor and/or each of the plurality of tissue anchors 321 by a frangible connection. In some embodiments, each of the at least one tissue anchor and/or each of the plurality of tissue anchors 321 may be releasably attached to an adjacent one of the at least one tissue anchor and/or each of the plurality of tissue anchors 321 by an adhesive connection. Other configurations are also contemplated. In some embodiments, each of the at least one tissue anchor and/or each of the plurality of tissue anchors 321 may be loaded on and/or may slide along a rail or another guide element disposed within the lumen 342 of the tubular sheath 340.

In some embodiments, the tissue anchor delivery device 310 may include a biasing mechanism (not shown) configured to advance the plurality of tissue anchors 321 distally within the tubular sheath 340 and/or along or relative to the elongate shaft 350. In some embodiments, the biasing mechanism may be configured to automatically advance the plurality of tissue anchors 321 distally within the tubular sheath 340 and/or along or relative to the elongate shaft 350 when a distalmost one of the plurality of tissue anchors 321 is deployed. In some embodiments, the biasing mechanism may be configured to manually advance the plurality of tissue anchors 321 distally within the tubular sheath 340 and/or along or relative to the elongate shaft 350 after the distalmost one of the plurality of tissue anchors 321 is deployed. In one example, the biasing mechanism may include a coil spring disposed within the tissue anchor delivery device 310 and/or the tubular sheath 340. Other biasing mechanisms are also contemplated—including but not limited to ratcheting mechanisms, push rods, etc.

In some embodiments, the tissue anchor delivery device 310 may include a stop feature (not shown) proximate and/or at the distal end of the tubular sheath 340. The stop feature may be configured to prevent distal advancement of the distalmost one of the plurality of tissue anchors 321 past a predetermined point. In at least some embodiments, the predetermined point may be the distal end of the tubular sheath 340. In some embodiments, the stop feature of the tissue anchor delivery device 310 may be similar in form and/or function to the stop feature 136 described above.

A method of treating soft tissue at the joint region (e.g., the shoulder 10) of the patient P may include creating the lateral portal 11 through the patient's skin to access the treatment site in the joint region (e.g., the shoulder 10), as seen in FIG. 1. In at least some embodiments, the superior portal 13 may be omitted. The method may include inserting the implant delivery device 40 through the lateral portal 11 in the joint region (e.g., the shoulder 10) to the treatment site, as seen in FIG. 2. In some embodiments, the method may include positioning the distal end of the implant delivery device 40 over the soft tissue (e.g., the tendon 24) and/or at the treatment site. The method may include deploying the sheet-like implant 12 from the implant delivery device 40 onto the soft tissue (e.g., the tendon 24) at the treatment site in the joint region (e.g., the shoulder 10) of the patient P. In some embodiments, the method may include securing the sheet-like implant 12 in place over the soft tissue (e.g., tendon 24) and/or at the treatment site with the tack member 84. In some embodiments, the method may include removing the implant delivery device 40 from the lateral portal 11 and/or the patient P while leaving and/or maintaining the sheet-like implant 12 in position over the soft tissue (e.g., the tendon 24) and/or at the treatment site.

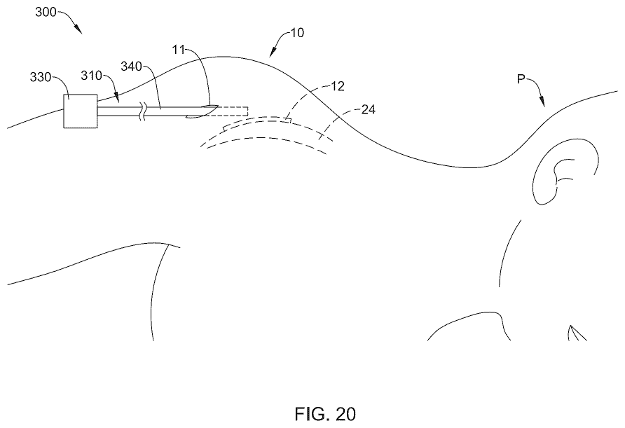

The method may include inserting the tissue anchor delivery device 310 into and/or through the lateral portal 11 in the joint region (e.g., the shoulder 10) to the treatment site, as seen in FIG. 20. In at least some embodiments, inserting the tissue anchor delivery device 310 into and/or through the lateral portal 11 to the treatment site may occur after removing the implant delivery device 40 from the lateral portal 11 and/or the patient P.

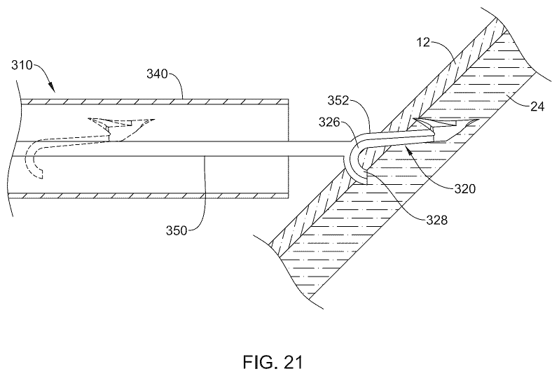

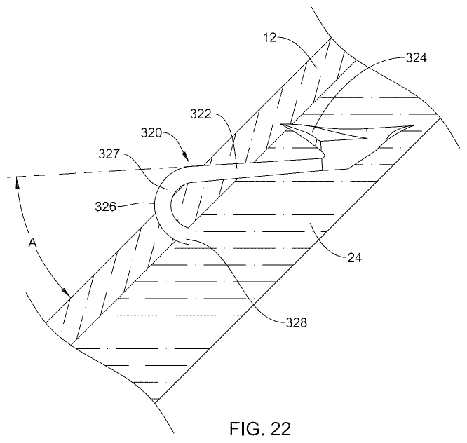

The method may include deploying at least one tissue anchor (e.g., the tissue anchor 320, the plurality of tissue anchors 321, etc.) from the tissue anchor delivery device 310 and/or the tubular sheath 340 into the sheet-like implant 12 and the soft tissue (e.g., the tendon 24) at the treatment site at an oblique angle A (e.g., FIG. 22) to an upper surface of the sheet-like implant 12 using the elongate shaft 350 in order to secure the sheet-like implant 12 to the soft tissue (e.g., the tendon 24), as seen in FIG. 21. As shown in FIG. 22, the oblique angle A may be measured between the upper surface of the sheet-like implant 12 and the single axial prong 322 of the tissue anchor 320. In some embodiments, the oblique angle A may be 60 degrees or less relative to the upper surface of the sheet-like implant 12. In some embodiments, the oblique angle A may be 45 degrees or less relative to the upper surface of the sheet-like implant 12. In some embodiments, the oblique angle A may be 30 degrees or less relative to the upper surface of the sheet-like implant 12.

In some embodiments, the free end 328 of the tissue securement arm 326 may be configured to penetrate into the sheet-like implant 12. Accordingly, in some embodiments, after deploying the at least one tissue anchor (e.g., the tissue anchor 320, the plurality of tissue anchors 321, etc.) from the tissue anchor delivery device 310 and/or the tubular sheath 340 into the sheet-like implant 12 and the soft tissue (e.g., the tendon 24) at the treatment site, the free end 328 of the tissue securement arm 326 may extend into and/or may penetrate the upper surface of the sheet-like implant 12, as seen in FIGS. 21-22.

In some embodiments, the method may include, after deploying the at least one tissue anchor (e.g., the tissue anchor 320, the plurality of tissue anchors 321, etc.) from the tissue anchor delivery device 310 and/or the tubular sheath 340 into the sheet-like implant 12 and the soft tissue (e.g., the tendon 24) at the treatment site, advancing the remaining tissue anchors of the plurality of tissue anchors 321 distally within the tubular sheath 340 to position the distalmost tissue anchor of the remaining tissue anchors of the plurality of tissue anchors 321 proximate the distal end of the tubular sheath 340 and/or to the predetermined point. The method may include engaging the distal tine 352 of the elongate shaft 350 with the distalmost tissue anchor of the remaining tissue anchors of the plurality of tissue anchors 321 proximate the distal end of the tubular sheath 340. The method may then include deploying the distalmost tissue anchor of the remaining tissue anchors of the plurality of tissue anchors 321 into the sheet-like implant 12 to secure the sheet-like implant 12 to the soft tissue (e.g., the tendon 24) at the treatment site using the elongate shaft 350. These steps may be repeated as necessary until all desired tissue anchors of the plurality of tissue anchors 321 have been deployed and/or until the sheet-like implant 12 is sufficiently secured to the soft tissue (e.g., the tendon 24) at the treatment site.

In some embodiments, the method may include inserting the bone anchor delivery device 90 into and/or through the lateral portal 11 to the treatment site. In some embodiments, the method may include deploying at least one bone anchor 92 from the bone anchor delivery device 90 into the sheet-like implant 12 to secure the sheet-like implant 12 to bone adjacent the treatment site. In some embodiments, the at least one bone anchor 92 may be deployed prior to deploying the at least one tissue anchor (e.g., the tissue anchor 320, the plurality of tissue anchors 321, etc.) from the tissue anchor delivery device 310 and/or the tubular sheath 340 into the sheet-like implant 12 and the soft tissue (e.g., the tendon 24) at the treatment site. In some embodiments, the at least one bone anchor 92 may be deployed subsequent to or after deploying the at least one tissue anchor (e.g., the tissue anchor 320, the plurality of tissue anchors 321, etc.) from the tissue anchor delivery device 310 and/or the tubular sheath 340 into the sheet-like implant 12 and the soft tissue (e.g., the tendon 24) at the treatment site.

After deploying the at least one bone anchor 92 and/or the at least one tissue anchor (e.g., the tissue anchor 320, the plurality of tissue anchors 321, etc.) from the tissue anchor delivery device 310 and/or the tubular sheath 340 into the sheet-like implant 12 and the soft tissue (e.g., the tendon 24) at the treatment site, the shoulder 10 of the patient P may resemble the views shown in FIG. 10 and FIG. 10A, except that the plurality of tissue anchors 130 of FIG. 10 would be replaced with the plurality of tissue anchors 321.

The materials that can be used for the various components of the medical devices, systems, and various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the tissue anchor(s), the bone anchor(s), the bone anchor delivery device, the tissue anchor delivery device, the tissue anchor deployment device, etc., and/or elements or components thereof.

In some embodiments, the system and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL®625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, portions or all of the system and/or components thereof may be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location of the system. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-protein and/or anti-bacterial agents (such as 2-methacryroyloxyethyl phosphorylcholine (MPC) and its polymers or copolymers); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A system for securing a sheet-like implant to soft tissue at a treatment site, comprising:
a tissue anchor delivery device including:
a tubular sheath defining a lumen; and
an elongate shaft extending through the lumen of the tubular sheath; and
a plurality of tissue anchors slidably disposed on the distal tine;
wherein a distalmost one of the plurality of tissue anchors is deployable from a distal end of the tubular sheath for insertion into the sheet-like implant and the soft tissue at the treatment site to secure the sheet-like implant to the soft tissue;
wherein each of the plurality of tissue anchors includes a single axial prong, at least one anchoring barb extending laterally from a distal portion of the single axial prong, and a tissue securement arm extending laterally from a proximal portion of the single axial prong.

2. The system of claim 1, wherein the elongate shaft is axially movable within the lumen of the tubular sheath.

3. The system of claim 2, wherein the tissue anchor delivery device includes a handle attached to a proximal end of the tubular sheath.

4. The system of claim 3, wherein actuation of the handle advances the plurality of tissue anchors distally.

5. The system of claim 1, further comprising a distal tine extending distally from the elongate shaft.

6. The system of claim 5, wherein the distal tine engages the tissue securement arm of the distalmost one of the plurality of tissue anchors.

7. The system of claim 1, wherein the at least one anchoring barb of each of the plurality of tissue anchors extends away from the elongate shaft in a first lateral direction and the tissue securement arm of each of the plurality of tissue anchors extends away from the elongate shaft in a second lateral direction, wherein the second lateral direction is opposite the first lateral direction.

8. The system of claim 1, wherein the plurality of tissue anchors are releasably attached together by a frangible connection

9. The system of claim 1, wherein the plurality of tissue anchors are releasably attached together by an adhesive connection.

10. A system for securing a sheet-like implant to soft tissue at a treatment site, comprising:
a tissue anchor delivery device including an elongate shaft and a distal tine extending distally from the elongate shaft; and
a tissue anchor releasably disposable on the distal tine;
wherein the tissue anchor is configured for insertion into the sheet-like implant and the soft tissue at the treatment site to secure the sheet-like implant to the soft tissue;
wherein the tissue anchor includes a single axial prong, at least one anchoring barb extending laterally from a distal portion of the single axial prong, and a tissue securement arm extending laterally from a proximal portion of the single axial prong.

11. The system of claim 10, wherein the tissue securement arm includes a curved portion extending away from the single axial prong to a free end, wherein the free end extends distal of a proximalmost extent of the curved portion.

12. The system of claim 11, wherein the free end of the tissue securement arm is configured to penetrate into the sheet-like implant.

13. The system of claim 10, wherein the tissue anchor is formed from a resorbable material.

14. The system of claim 10, wherein the tissue anchor delivery device includes a handle at a proximal end thereof, wherein actuation of the handle advances the tissue anchors distally.

15. The system of claim 10, wherein the at least one anchoring barb of the tissue anchor extends away from the elongate shaft in a first lateral direction and the tissue securement arm of the tissue anchor extends away from the elongate shaft in a second lateral direction, wherein the second lateral direction is opposite the first lateral direction.

16. The system of claim 10, wherein the distal tine engages the tissue securement arm of the tissue anchor.

17. A system for securing a sheet-like implant to soft tissue and/or bone at a treatment site, comprising:
a tissue anchor delivery device including an elongate shaft having a longitudinal axis and a plurality of tissue anchors movably disposed within the elongate shaft; and
a tissue anchor deployment device separate from the tissue anchor delivery device, the tissue anchor deployment device oriented non-parallel to the elongate shaft of the tissue anchor delivery device and configured to engage a first tissue anchor of the plurality of tissue anchors proximate a distal end of the elongate shaft to insert the first tissue anchor into the sheet-like implant and the soft tissue and/or bone at the treatment site to secure the sheet-like implant thereto.

18. The system of claim 17, wherein the first tissue anchor is exposed to an exterior of the elongate shaft proximate the distal end of the elongate shaft.

19. The system of claim 17, wherein the tissue anchor deployment device includes an outer tube configured to engage the elongate shaft and an inner member configured to engage the first tissue anchor distal of the distal end of the elongate shaft.

20. The system of claim 17, wherein the tissue anchor deployment device includes an outer tube configured to engage the elongate shaft and an inner member configured to engage the first tissue anchor through a side facing window formed through a wall of the elongate shaft.
